(19) United States
(12) Patent Application Publication
(10) Pub. No.: US 2026/0224240 A1
(43) Pub. Date: Aug. 6, 2026

(54) SELF-PULLING OCCLUSION CROSSING CATHETER WITH CONTROLLED STEERING BASED ON IMPEDANCE SENSING

(71) Applicant: KONINKLIJKE PHILIPS N.V., EINDHOVEN (NL)

(72) Inventors: Bernardus Hendrikus Wilhelmus HENDRIKS, EINDHOVEN (NL); Vipul Shrihari PAI RAIKAR, SOMERVILLE, MA (US); Leili SALEHI, WALTHAM, MA (US); Alyssa TORJESEN, EXETER, NH (US); René Leonardus Jacobus Marie UBACHS, EINDHOVEN (NL)

(21) Appl. No.: 19/143,436

(22) PCT Filed: Dec. 21, 2023
(86) PCT No.: PCT/EP2023/087253

§ 371 Date: Jun. 26, 2025

Related U.S. Application Data
(60) Provisional application No. 63/436,901, filed on Jan. 4, 2023.

(51) Int. Cl. A61B 17/3207; A61B 17/00; A61B 17/22

(52) U.S. Cl. CPC A61B 17/32075; A61B 17/00234; A61B 17/22; A61B 2017/00026; A61B 2017/00061; A61B 2017/00199; A61B 2017/00305; A61B 2017/00398; A61B 2017/00778; A61B 2017/22075; A61B 2017/22077; A61B 2017/22094

(57) ABSTRACT

An intravascular therapy device (10) includes a catheter (12); and a plurality of mutually parallel cutting instruments (14, 16, 18) disposed at least at a distal end of the catheter. Each cutting instrument is configured to engage a portion of a clot disposed in a blood vessel into which the catheter is advanced. Each cutting instrument is independently advanceable into the clot.

### FIELD

The following relates generally to the catheter arts, mechanical thrombectomy arts, spectroscopy arts, and related arts.

### BACKGROUND

A chronic total occlusion (CTO) is a complete obstruction of a blood vessel. To treat such a CTO (or other nearly complete occlusion) in a typical intervascular therapy workflow, a guidewire is initially inserted into the blood vessel and manipulated to cross the occlusion (sometimes also referred to herein as a clot). After crossing the occlusion, an interventional catheter (i.e., a balloon/stent device, etc.) is inserted along the guidewire to access the CTO to perform the treatment. Since the guidewire is flexible, passing it through the CTO is difficult and sometimes not possible because of buckling issues with the guidewire. The problem is that the guidewire is not stiff enough to push it across the CTO without buckling. Furthermore, the guidewire should stay inside the blood vessel, hence steerability is required during advancing through the CTO, otherwise the guidewire may be inadvertently pushed into or even through the blood vessel wall, creating undesirable damage to or rupture of the blood vessel. In order to know whether the guidewire or catheter is inside the (blocked) lumen, inside blood vessel tissue, or has fully punctured the vessel wall, sensing is beneficial to provide feedback.

One way to obtain such feedback is by employing interventional imaging using a modality such as computed tomography (CT) or another X-ray imaging modality, or ultrasound imaging. However, while these imaging techniques can be useful in tracking progress of the guidewire to the occlusion, they have limited spatial resolution and contrast, and hence may be unable to accurately detect blood vessel penetration as the operator attempts to engage the tip of the guidewire into the occlusion. An experienced operator may be able to judge the tissue being penetrated by manual tactile feedback as he or she pushes the guidewire into the occluded region, and/or by monitoring aspirated material if aspiration is performed during the crossing, but these can be also inaccurate and mislead the operator.

The following discloses certain improvements to overcome these problems and others.

### SUMMARY

In some embodiments disclosed herein, an intravascular therapy device includes a catheter; and a plurality of mutually parallel cutting instruments disposed at least at a distal end of the catheter. Each cutting instrument is configured to engage a portion of a clot disposed in a blood vessel into which the catheter is advanced. Each cutting instrument is independently advanceable into the clot.

In some embodiments disclosed herein, an intravascular therapy device includes a flexible catheter including at least three mutually parallel flexible sub-catheters. Each sub-catheter has a tip configured to engage a portion of a blood clot. Each sub-catheter is independently movable respective to the other sub-catheters of the least three mutually parallel flexible sub-catheters.

In some embodiments disclosed herein, an occlusion crossing method includes performing an electrical spectroscopy measurement on at least one of cutting instruments disposed at least at a distal end of a catheter to engage a portion of a clot disposed in a blood vessel into which the catheter is advanced; and based on the electrical spectroscopy measurement, determining a type of tissue with which the at least one cutting instrument is engaged.

One advantage resides in providing feedback to correct an advancement of a catheter into an occluded region.

Another advantage resides in providing a catheter that provides a pulling force component to improve the ability and efficiency of penetration through an occlusion during crossing of the occlusion.

Another advantage resides in providing a catheter with multiple cutting instruments (e.g. sub-catheters) to engage an occlusion.

Another advantage resides in measuring an impedance of cutting instruments of catheter to determine a type of tissue with which the catheter is engaged.

Another advantage resides in providing for optical measurement of a type of tissue with which the catheter is engaged.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIGS. 1 and 2 diagrammatically illustrate two embodiments of an intravascular therapy device in accordance with the present disclosure.

FIG. 3 diagrammatically illustrates a method of performing an intravascular therapy method using the device of FIG. 1.

### DETAILED DESCRIPTION

In embodiments disclosed herein, a catheter (e.g., a guidewire in some embodiments) provides a pulling force to assist in penetrating through an occlusion to achieve crossing of the occlusion. This pulling force advantageously can reduce or eliminate the use of a pushing force to achieve crossing. As previously noted, attempting to cross an occlusion by pushing a catheter or guidewire through the occlusion can lead to buckling of the catheter and inability or difficulty in achieving the crossing. This is because the pushing force is applied at the proximal end of the catheter, that is, the end located outside of the patient's vasculature, and the pushing force transmits along the entire length of the catheter disposed in the vasculature to reach the distal end thereof which is engaged with the occlusion. As the catheter is flexible to accommodate tortuous curves of the vasculature through which it passes, the transmitted pushing force can lead to buckling of the catheter. By contrast, catheters disclosed herein employ a plurality of mutually parallel cutting instruments disposed at least at a distal end of the catheter (and in some embodiments, comprising sub-catheters running the length of the catheter or other catheter. Each of the mutually parallel cutting instruments can move or slide relative to the other cutting instruments. If there are at least three cutting instruments, then the one cutting instrument that is currently moving produces less frictional force than the remaining two (or more) cutting instruments that are not currently moving. Cf. Scali et al., “Ovipositor-inspired steerable needle: design and preliminary experimental evaluation,” 2018 Bioinspir. Biomim. 13 016006. Consequently, the nonmoving cutting instruments provide a frictional force that tends to hold the tip of the catheter in a fixed position so as to allow the one moving cutting instrument to advance into the clot. This process is cyclically repeated for each cutting instrument in turn to advance the tip of the catheter as a whole through the clot, thereby achieving (or at least assisting in achieving) crossing of the clot. In various embodiments, the cyclical advancement of each cutting instrument in turn can be achieved manually (e.g. by a human operator successively pushing each cutting instrument comprising a sub-catheter in turn), or by a mechanical or electromechanical mechanism.

A further advantage of illustrative catheters disclosed herein is that the cutting instruments provide a platform for performing tissue measurements. In one approach, each of the cutting instruments are electrically conductive, and are used as electrodes in an impedance measurement (or other type of electrical characteristic measurement). For example, a spectral impedance measurement between a pair of the cutting instruments can provide a tissue signature for identifying the type of tissue (e.g., blood, clot tissue, blood vessel wall, et cetera) disposed between the pair of cutting instruments. See, e.g. Gabriel et al., “The dielectric properties of biological tissues: III. Parametric models for the dielectric spectrum of tissues,” Phys. Med. Biol. vol. 41 pp. 2271-93 (1996); Ambrogio et al., “Investigation of Blood Coagulation Using Impedance Spectroscopy: Toward Innovative Biomarkers to Assess Fibrinogenesis and Clot Retraction”, Biomedicines 2022, 10, 1833.

In another approach, the cutting instruments comprise sub-catheters each including one or more optical fibers (or waveguides) for injecting light to the distal tips of the sub-catheters. By arranging the optical fiber ends of neighboring cutting instruments to face each other, light can be transmitted through the gap between the neighboring cutting instruments so as to perform an optical measurement of the tissue disposed between the cutting instruments. Optical spectroscopy can thereby be performed to provide a tissue signature for identifying the type of tissue (e.g., blood, clot tissue, blood vessel wall, et cetera) disposed between the pair of cutting instruments. See, e.g. Skyrman et al., “Clot composition characterization using diffuse reflectance spectroscopy in acute ischemic stroke,” Biomedical Optics Express vol. 13 no. 6 (June 2022); Skyrman et al., “Identifying clot composition using intravascular diffuse reflectance spectroscopy in a porcine model of endovascular thrombectomy”, J. NeuroIntervent Surg April 2021. In some embodiments, a pair (or more) fibers can be included. One fiber can be used to emit light, and another fiber can be used to receive light.

Although described primarily in terms of spectral impedance measurements, the systems and methods described herein can also include optical sensing (such as optical spectroscopy, fluorescence spectroscopy, Raman spectroscopy, optical coherence tomography etc.) requiring an optical fiber), pressure sensors in a sub-catheter (i.e., to measure the resistance of the tissue), acoustic sensors (i.e., the measure the acoustic properties), and so forth. Other types of sensors that include the cutting instruments are also contemplated for sensing the type of tissue with which the cutting instruments are engaged.

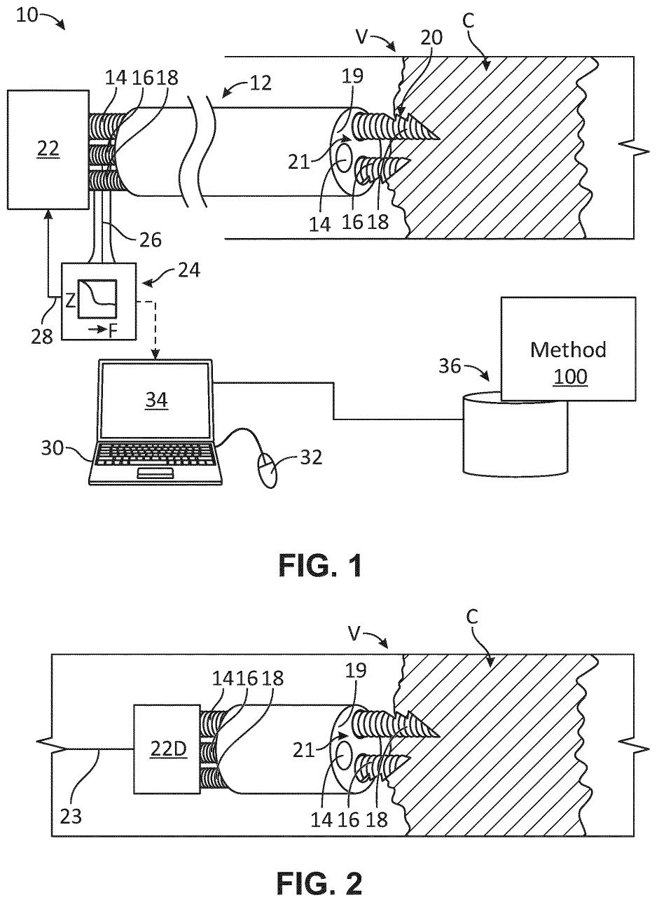

With reference to FIG. 1, an intravascular therapy device 10 for treating a clot C or an occlusion (e.g., a CTO or nearly complete occlusion) in a blood vessel V is diagrammatically shown. As used herein, “clot” and “occlusion” are synonymous, referring to a complete, or nearly complete, blockage of the flow of blood through a blood vessel. The therapy device 10 includes a flexible catheter (or guidewire) 12 advanced into the vessel V and adjacent the clot C. The flexible catheter 12 is flexible in the sense that the catheter 12 can be pushed through a tortuous vascular path to move its distal end to the clot C with the flexible catheter 12 bending or flexing during the insertion process to conform with the tortuous vascular path. The catheter 12 may, for example, be a guidewire in some embodiments. In a first embodiment shown in FIG. 1, A plurality of mutually parallel cutting instruments 14, 16, 18 in this embodiment comprise sub-catheters of the catheter 12 that extend to a distal end of the catheter 12 (i.e., adjacent or near the clot C). FIG. 1 shows three mutually parallel cutting instruments 14, 16, 18; although any suitable number of at least three cutting instruments can be implemented. Each cutting instrument 14, 16, 18 is independently advanceable into the clot C. Each cutting instrument 14, 16, 18 is configured to engage a portion of the clot C, for example by having tapered or pointed tips. The cutting instruments 14, 16, 18 of the catheter 12 can be used to create a pulling force to pull the catheter 12 through the clot C to thereby cross the clot C.

After the catheter 12 crosses the clot C, various types of therapy can be applied. In some embodiments, the illustrative catheter 12 is a guidewire. In such embodiments, after the guidewire 12 crosses the clot 12, a second catheter (not shown) is inserted along the guidewire 12 by inserting the proximal end of the guidewire 12 into a lumen of the second catheter, so that the guidewire 12 can guide the distal end of the second catheter up to (and possibly a short distance past) the clot C. The second catheter suitably carries an angioplasty balloon, a deployable stent, a mechanical or laser cutter, and/or other therapy component to treat the clot C by angioplasty, stenting, thrombectomy, or so forth.

In other contemplated embodiments, the illustrated catheter 12 may be the second catheter that is inserted along a previously inserted (and much smaller-diameter) guidewire via a guidewire lumen 21. In this case, the illustrated catheter 12 suitably carries the therapy component. while the guidewire be used to both cross the clot C as disclosed herein and to also carry a therapy component (not shown) for treating the clot C.

The sub-catheters 14, 16, 18 of the catheter 12 are held together in a mutually parallel bundle by a suitable retention mechanism 19. In the illustrative example, this retention mechanism comprises a carrier catheter 19 with lumens inside which the sub-catheters 14, 16, 18 are disposed. In another contemplated embodiment, the retention mechanism may include interlocking mechanisms built into the sub-catheters 14, 16, 18 themselves, such as mating longitudinal keyed edges and slots (not shown) of the sub-catheters 14, 16, 18 that engage to lock the sub-catheters 14, 16, 18 into the mutually parallel arrangement. In either case, the retention mechanism 19 allows the sub-catheters 14, 16, 18 to move or slide relative to one another, and more particularly to allow one of the sub-catheters (e.g. sub-catheter 18) to so move or slide at any given time while the other sub-catheters (e.g. sub-catheters 14, 16) may be fixed or may not be fixed. Such movement is done cyclically, to successively advance each sub-catheter in turn, e.g. advance and then retract sub-catheter 18, then advance and then retract sub-catheter 14, then advance and then retract sub-catheter 16, and then advance and then retract sub-catheter 18, and so forth. In another example, each of the sub-catheters 14, 16, 18 can be advanced, then the retention mechanism 19 can be pulled forward to retract the sub-catheters 14, 16, 18. This process can be repeated as needed. In another example, the catheter 12 can be steered so that each of the sub-catheters 14, 16, 18 can be advanced in different movements. These are merely examples and should not be construed as limiting.

For illustration, as shown in FIG. 1, a first cutting instrument 14 is advanced out of the catheter 12 and engaged with a portion of the clot C. A second cutting instrument 16 is partially advanced out of the catheter 12. A third cutting instrument 18 is shown disposed within the catheter 12. Each of the cutting instruments 14, 16, 18 includes serrations 20, such as an illustrative serrations or a sawtooth structure 20, configured to engage a portion of the clot C. The serrations 20 can anchor the extended end of the sub-catheter 18 in the clot C so that when the extended sub-catheter 18 is then withdrawn the serrations 20 anchor the sub-catheter 18 to facilitate pulling the remainder of the catheter 12 deeper into the clot C. This mechanism for pulling the catheter 12 into the clot C to traverse it has some similarity to the biomechanical mechanism by which the ovipositor of a parasitoid wasp penetrates into a host to deposit eggs. Cf. Scali et al., “Ovipositor-inspired steerable needle: design and preliminary experimental evaluation,” 2018 Bioinspir. Biomim. 13 016006.

The catheter 12 can include other components, such as an illustrated central lumen 21 for aspiration or for receiving a guidewire (if the catheter 12 is not itself a guidewire) or so forth. As another example, the catheter 12 may carry a therapy device (not shown-e.g. angioplasty balloon, stent delivery device, cutting tool, et cetera).

In an optical embodiment, the electronic processing device 24 includes a light source (not shown) coupled to send light into the optical fiber of a first cutting instrument 16 and an optical sensor (not shown) coupled to receive the light from the optical fiber of a second cutting instrument 16 after the light passes out an aperture at the distal end of the first cutting instrument 14 and into an aperture at the distal end of the second cutting instrument. The light is suitably multispectral to enable measurement of an optical spectrum which serves as a signature of the material disposed between the cutting instruments of the pair, enabling accurate determination of whether that tissue is clot material, blood vessel wall material, or blood, for example.

In another example, the electronic processing device 24 can be include a fluorophore (or molecular marker) delivery device configured to deliver a compound in the vessel V to enable imaging of the vessel V, the clot C, and/or a portion of the catheter 12 including the sub-catheters 14, 16, 18. In some examples, an autofluorescence process can be induced by the presence of collagen and elastin in the wall of the vessel V. In another example, the cutting instruments 14, 16, 18 comprise ultrasound sensors, and the tissue determination can be based on ultrasound measurements. In general the sensor can comprise at least two of the plurality of mutually parallel cutting instruments 14, 16, 18, with the sensor configured to sense a type of tissue with which the at least two of the plurality of mutually parallel cutting instruments are engaged. This arrangement advantageously leverages the two cutting instruments to provide a path through the tissue, e.g. as two electrodes contacting across the tissue between the cutting instruments for electrical tissue measurement, or similarly providing optical aperture/collector for optical tissue measurement. Even more broadly, in some embodiments the sensor can comprise at least one of the plurality of mutually parallel cutting instruments 14, 16, 18, with the sensor configured to sense a type of tissue with which the cutting instrument is engaged. For example, two electrodes for an electrical tissue measurement could be integrated into a single cutting element, or an optical aperture and collector could be integrated into a single cutting element.

In addition to facilitating advancement of the catheter 12 through the clot C, the sub-catheters 14, 16, 18 can also serve as components of a sensor for detecting the type of tissue into which the tip of the catheter 12 is engaged. For example, the cutting instruments 14, 16, 18 may each comprise an optical fiber extending along the length thereof to enable optical measurements, and/or can each be made of an electrically conducting material (e.g. a metal) to enable electrical measurements. In a particular example, the measurement can comprise an electrical impedance measurement. The cutting instruments 14, 16, 18 are connected to a motor 22 operably connected to cyclically advance each of the cutting instruments 14, 16, 18 in turn through the catheter 12 and into the clot C. For example, the motor 22 can drive a cam mechanism to which proximal ends of the cutting instruments 14, 16, 18 are secured. As the cam rotates it successively pushes each successive cutting instrument forward then withdraws it. The cutting instruments 14, 16, 18 are also connected to an electronic processing device 24 (such as a workstation computer, a tablet, or more generally a computer) via wires 26 to implement a tissue sensor (not shown).

The electronic processing device 24 is, in an electrical sensing embodiment, operatively connected to measure an A.C. electrical characteristic (e.g. impedance) as a function of frequency of the applied electrical current between a pair of the cutting instruments 14, 16, 18 or between one of the cutting instruments and electrical ground, a counter-electrode, or another electrical reference (not shown). The resulting impedance spectrum then serves as a signature of the material disposed between the cutting instruments of the pair, enabling accurate determination of whether that tissue is clot material, blood vessel wall material, or blood, for example.

The motor 22 in some embodiments is also operably connected to the electronic processing device 24 via a wires 28, and the electronic processing device 24 is configured to control the motor 22 to perform the advancement of the cutting instruments 14, 16, 18.

The electronic processing device 24 includes an electronic processor 30 (e.g., a microprocessor), optionally at least one user input device (e.g., a mouse, a keyboard, a trackball, and/or the like) 32, and a display device 34 (e.g., an LCD display, plasma display, cathode ray tube display, and/or so forth) for displaying the results of the electrical or optical tissue measurement.

The electronic processor 30 is operatively connected with one or more non-transitory storage media 36. The non-transitory storage media 36 may, by way of non-limiting illustrative example, include one or more of a magnetic disk or other magnetic storage medium; a solid-state drive, flash drive, or other electronic memory; an optical disk or other optical storage; various combinations thereof; or so forth. It is to be understood that any reference to a non-transitory medium or media 36 herein is to be broadly construed as encompassing a single medium or multiple media of the same or different types. Likewise, the electronic processor 30 may be embodied as a single electronic processor or as two or more electronic processors. The non-transitory storage media 36 stores instructions executable by the at least one electronic processor 30.

In the embodiment of FIG. 1, the cutting instruments 14, 16, 18 are in the form of sub-catheters 14, 16, 18 each extending along the length of the catheter 12 so that their distal ends are disposed at the distal end of the catheter 12. This design enables the motor 22 that operates the cutting instruments 14, 16, 18 to be located at the proximal end of the catheter 12 outside of the patient.

FIG. 2 shows another embodiment of the vascular therapy device 10, in which the cutting instruments 14, 16, 18 are not sub-catheters, but instead are located only at the distal end of the catheter 12. The embodiment of FIG. 2 is configured similarly to the embodiment of FIG. 1. However, here the cutting instruments 14, 16, 18 are disposed only at the distal end of the catheter 12 and are not in the form of sub-catheters extending the length of the catheter 12. To enable moving the cutting instruments 14, 16, 18 back and forth in this embodiment, a cam mechanism or other drive mechanism 22D is positioned at the distal end of the catheter 12 to drive the reciprocating motion of the cutting instruments 14, 16, 18. A coupling 23 connects the distally located drive mechanism 22D to a power source located at the proximal end of the catheter 12, outside the patient. In one approach, the drive mechanism 22D includes an electrical motor, in which case the coupling 23 is suitably a pair of electrical wires delivering electrical power to the motor. In another approach, the motor is located at the proximal end of the catheter 12, and the coupling 23 is a mechanical coupling such as a wire with high torsional resistance that delivers rotational force from the motor to the drive mechanism 22D. If electrical (e.g. impedance) spectroscopy measurements are to be made, the coupling 23 may further include wires connected with the respective cutting instruments 14, 16, 18 to enable connection with an impedance meter of the electronic processing device 24. Conversely, if optical spectroscopy measurements are to be made, the coupling 23 may further include optical fibers connected with the respective cutting instruments 14, 16, 18 to enable optical signals to be transmitted to and from the cutting instruments 14, 16, 18.

The at least one electronic processor 30 is configured as described above to perform an occlusion crossing method or process 100. The non-transitory storage medium 36 stores instructions which are readable and executable by the at least one electronic processor 30 to perform disclosed operations including performing the occlusion crossing method or process 100. In some examples, the method 100 may be performed at least in part by cloud processing.

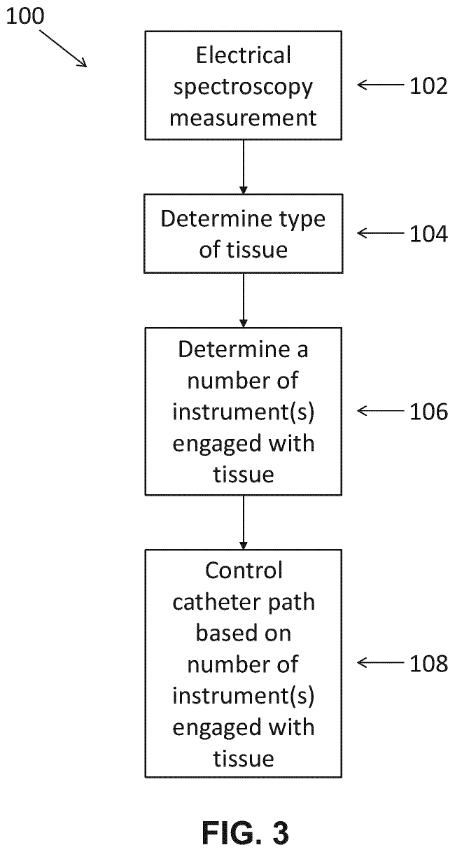

Referring to FIG. 3, and with continuing reference to FIG. 1 (although the method 100 is applicable to the embodiment of the device 10 shown in FIG. 2), an illustrative embodiment of the occlusion crossing method 100 is diagrammatically shown as a flowchart. To begin the method 100, the catheter 12 is inserted into the blood vessel V and adjacent the clot C.

At an operation 102, an electrical spectroscopy measurement is performed on at least one of the cutting instruments 14, 16, 18. In some examples, the electrical spectroscopy measurement can be an impedance measurement of the cutting instrument(s) 14, 16, 18. The output is an impedance spectrum or a part of an impedance spectrum, i.e. the impedance as a function of AC frequency. In another embodiment, the operation 102 may be an optical spectroscopy measurement producing an optical spectrum. In another embodiment, the impedance can be measured at a certain frequency (or only a few separate frequencies) and the determination of the tissue can be performed based on these measurements.

At an operation 104, based on the electrical (or optical) spectroscopy measurement, a type of tissue with which the at least one cutting instrument 14, 16, 18 is engaged is determined. For example, an electrical (or optical) spectroscopy measurement for the at least one cutting instrument 14, 16, 18 being engaged with the clot C will be different from an electrical spectroscopy measurement for the at least one cutting instrument 14, 16, 18 being engaged with healthy tissue (i.e., the vessel V).

At an operation 106, a number of the plurality of cutting instruments 14, 16, 18 engaged with the clot C based on the determined type of tissue is determined. For example, the electrical spectroscopy measurement can determine that two of the cutting instruments 14, 16, 18 are engaged with the clot C, and the other of the cutting instruments 14, 16, 18 is not engaged with the clot C.

At an operation 108, a path of the catheter 12 relative to the clot C can be controlled based on the determined type of tissue with which the cutting instrument(s) 14, 16, 18 is engaged. Using the same example, the path of the catheter 12 can be controlled by the motor 22 so that the cutting instrument 14, 16, 18 that is not engaged with the clot C can then be engaged with the clot C. Advantageously, the use of at least three cutting instruments 14, 16, 18 facilitates such steering of the penetration. For example, if one cutting instrument is reciprocated more frequently than the other two this can tilt the direction of penetration.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An intravascular therapy device comprising:
a catheter; and
a plurality of mutually parallel cutting instruments disposed at least at a distal end of the catheter, each cutting instrument configured to engage a portion of a clot disposed in a blood vessel into which the catheter is advanced, each cutting instrument being independently advanceable into the clot.

2. The intravascular therapy device of claim 1, wherein the plurality of cutting instruments each includes serrations configured to engage a portion of the clot.

3. The intravascular therapy device claim 1, wherein the plurality of cutting instruments each comprise an optical fiber.

4. The intravascular therapy device of claim 1, wherein the plurality of cutting instruments each are made from an electrically conducting material.

5. The intravascular therapy device of claim 1, further including at least one electronic processoroperatively connected to measure an electrical characteristic between a pair of the cutting instruments or between one of the cutting instruments and an electrical reference, the electronic processor programmed to:
perform an electrical spectroscopy measurement on at least one of the cutting instruments; and
based on the electrical spectroscopy measurement, determine a type of tissue with which the at least one cutting instrument is engaged.

6. The intravascular therapy device of claim 5, wherein the least one electronic processor is programmed to:
determine a number of the plurality of cutting instruments engaged with the clot based on the determined type of tissue.

7. The intravascular therapy device of claim 5, wherein the least one electronic processor is programmed to:
control a path of the catheter relative to the clot based on the determined type of tissue that the at least one cutting instrument is engaged with.

8. The intravascular therapy device of claim 5, wherein the electrical spectroscopy measurement comprises an impedance measurement.

9. The intravascular therapy device of claim 1, further including:
a motor operably connected to cyclically advance each of the cutting instruments of the plurality of cutting instruments in turn.

10. The intravascular therapy device of claim 1, wherein the plurality of mutually parallel cutting instruments comprises at least three mutually parallel cutting instruments.

11. The intravascular therapy device of claim 1, wherein the plurality of mutually parallel cutting instruments comprises a plurality of mutually parallel sub-catheters, each sub-catheter extending along a length of the catheter and having a distal end configured to engage the portion of the clot disposed in the blood vessel into which the catheter is advanced.

12. The intravascular therapy device of claim 1, further comprising:
a sensor comprising at least one of the plurality of mutually parallel cutting instruments the sensor the configured to sense a type of tissue with which the at least one of the plurality of mutually parallel cutting instruments are engaged.

13. An intravascular therapy device comprising:
a flexible catheter including at least three mutually parallel flexible sub-catheters wherein each sub-catheter has a tip configured to engage a portion of a blood clot;
and
wherein each sub-catheter is independently movable respective to the other sub-catheters of the least three mutually parallel flexible sub-catheters.

14. The intravascular therapy device of claim 13, wherein the tip of each sub-catheter has serrations on an outer surface of the tip.

15. The intravascular therapy device of claim 13, wherein at least two sub-catheters include an optical fiber running therethrough and having optically coupled apertures at the tips of the at least two sub-catheters.

16. The intravascular therapy device of claim 13, wherein the sub-catheters comprise an electrically conductive material.

17. An occlusion crossing method comprising:
performing an electrical spectroscopy measurement on at least one of cutting instruments disposed at least at a distal end of a catheter to engage a portion of a clot disposed in a blood vessel into which the catheter is advanced; and
based on the electrical spectroscopy measurement, determining a type of tissue with which the at least one cutting instrument is engaged.

18. The method of claim 17, further including:
determining a number of the plurality of cutting instruments engaged with the clot based on the determined type of tissue.

19. The method claim 17, wherein the least one electronic processor is programmed to:
controlling a path of the catheter relative to the clot based on the determined type of tissue that the at least one cutting instrument is engaged with.

20. The method of claim 17, wherein the electrical spectroscopy measurement comprises an impedance measurement.
